(19) United States
(12) Patent Application Publication
(10) Pub. No.: US 2010/0173856 A1
(43) Pub. Date: Jul. 8, 2010

(54) METHODS OF PRODUCING AND USING NUTRITIONAL AND PHARMACEUTICAL COMPOSITIONS THAT INCLUDE ONE OR MORE ACTIVE SUBSTANCES

(75) Inventor: Erwin Denk, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Olive Denk, Inc., San Diego, CA (US)

(21) Appl. No.: 12/728,992

(22) Filed: Mar. 22, 2010

Related U.S. Application Data
(63) Continuation of application No. 11/864,809, filed on Sep. 28, 2007.
(60) Provisional application No. 60/848,286, filed on Sep. 28, 2006.

(51) Int. Cl. A61K 31/222; A61K 31/7034; A61K 31/351; A61P 29/00

(52) U.S. Cl. 514/25; 514/549; 514/460

(57) ABSTRACT

Methods for producing a composition that includes an active agent, nutraceutical and pharmaceuticals compositions, and methods for ameliorating and/or treating a disease using the composition that includes an active substance are described herein. More particularly, methods for producing a composition that contains one or more active substances such as oleocanthal, pharmaceutical compositions that include one or more active substances such as oleocanthal, and methods of ameliorating and/or treating inflammation using compositions that contains one or more active substances such as oleocanthal are described.

### CROSS-REFERENCE TO RELATED APPLICATIONS

This application is a continuation of U.S. patent application Ser. No. 11/864,809, filed Sep. 28, 2007, which claims priority to U.S. Provisional Application No. 60/848,286 filed on Sep. 28, 2006, entitled METHODS OF PRODUCING AND USING PHARMACEUTICAL COMPOSITIONS THAT INCLUDE ONE OR MORE ACTIVE SUBSTANCES; which applications both are incorporated herein by reference in their entireties.

### BACKGROUND OF THE INVENTION

1. Field of the Invention

This invention relates to the fields of nutraceutical substances, and pharmaceutical chemistry and medicine. In particular, it relates to methods for producing a composition that includes an active agent, pharmaceuticals compositions, and methods for ameliorating and/or treating a condition or a disease using the composition that includes an active substance. More particularly, it relates to methods for producing a composition that contains one or more active substances such as, for example, oleocanthal, nutraceutical and/or pharmaceutical compositions that include one or more active substances such as oleocanthal, and methods of ameliorating and/or treating various conditions, including inflammation using a composition that contains one or more active substances such as oleocanthal.

2. Description of the Related Art

Inflammation is the body's basic way of reacting to infection, irritation or other injury. In some instances, the body inappropriately triggers an inflammatory response despite the lack of infection and/or injury. In an effort to protect itself, the body releases white blood cells into the tissue in an attempt to rid the body of the foreign substance such as bacteria and/or cellular debris, and increases blood flow to the area. Typical symptoms of inflammation include but are not limited to redness, joint pain, joint stiffness, swelling, pain, chills, headaches, fever, and/or fatigue. Free radicals are highly chemically reactive atom, molecules or molecular fragments with a free or unpaired electron. Free radicals can damage important cellular molecules such as DNA or lipids or other parts of the cell. That damage can lead to various diseases or adverse biological conditions.

Many current medications that are used for inflammation or to inhibit or counteract the effects of free radicals come with adverse side effects.

There exists a need for additional medications and substances for ameliorating or inhibiting inflammation and pain, free radical conditions, and for materials without the adverse side effects associated with current medications and substances.

### SUMMARY OF THE INVENTION

One embodiment described herein relates to a composition for ameliorating or inhibiting inflammation or pain, or compositions for reducing free radicals or cellular oxidation. In some embodiments the compositions can be derived from olives, and can include, for examples, substances such as oleocanthal, as well as additional active or inactive substances. Another embodiment disclosed herein relates to a process for producing such compositions that contains one or more active substances such as oleocanthal. Still another embodiment described herein relates to a method for ameliorating or treating inflammation that includes administering to a subject in need such compositions.

### BRIEF DESCRIPTION OF THE DRAWINGS

FIG. 1 shows a chromatographic spectrum of the composition obtained from the process described herein.

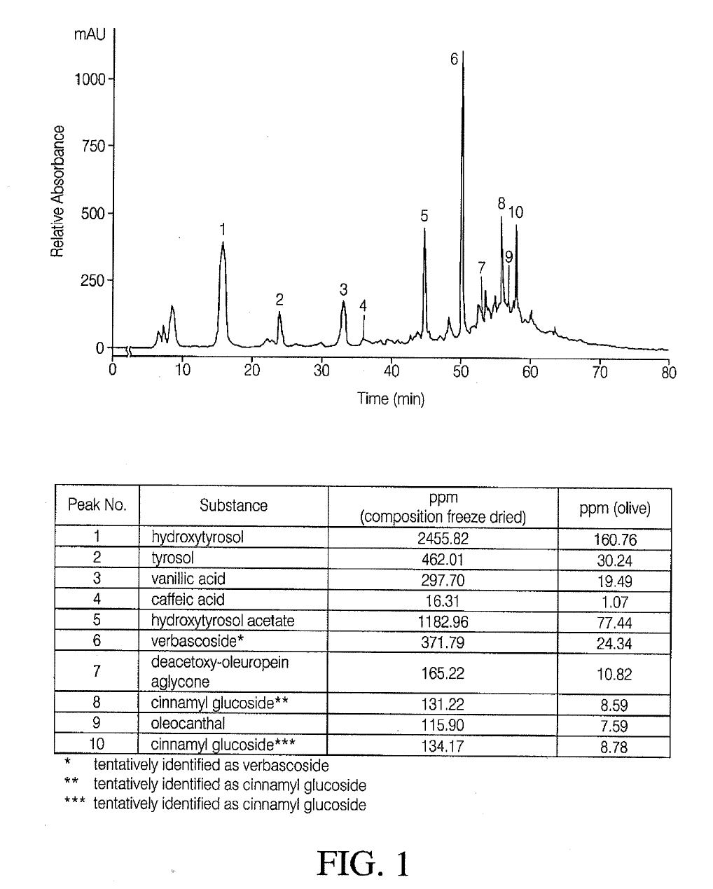

### DETAILED DESCRIPTION OF THE PREFERRED EMBODIMENT

The disclosed invention relates to methods and compositions for decreasing inflammation. Also, some embodiments relate to methods and compositions for providing an antioxidant to a subject and for decreasing oxidation and/or free radicals in an animal. The methods and compositions can thus be used to ameliorate inflammation, cardiovascular disease, high cholesterol levels, gastrointestinal. Disorders and blood sugar related disorders.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the invention herein described.

The typical medications used to treat inflammation and pain include NSAIDs, corticosteroids such as prednisone, and anti-malarial medication (e.g., hydroxychloroquine). Some embodiments of the instant invention relate to new compositions for treating inflammation and pain. In preferred embodiments the compositions can be made from olives. The compositions can include various substances, including for example, oleocanthal. Oleocanthal, (E)-4-hydrdoxyphenethyl-4-formul-3-(2-oxoethyl)hex-4-enoate, is a chemical found in olives. Recently, scientists have discovered that oleocanthal exhibits similar activity as the currently available NSAID, ibuprofen. However, no one has been able to produce a composition from olives that contains one or more active substances such as oleocanthal as a composition that can be formulated into a pharmaceutical composition such as a tablet.

Some embodiments disclosed herein relate to compositions. For example, the compositions can be derived from olives. The compositions can include one or more of a variety of substances and described below or the compositions can be made partially or completely by synthetic or other combinatorial methods. For example, the compositions can include oleocanthal and at least one other compound such as a phytochemical, an antioxidant, and/or a secoiridoid. Other embodiments disclosed herein relate to a composition that includes at least two, three, four, five, six, seven, eight, or nine of the compounds selected from the group consisting of oleocanthal, hydroxytyrosol, tyrosol, vanillic acid, caffeic acid, hydroxytyrosol acetate, verbascoside, deacetoxy-oleuropein aglycone, and cinnamyl glucoside. Still other embodiments disclosed herein related to a composition that includes oleocanthal and at least one other compound selected from the group consisting of hydroxytyrosol, tyrosol, vanillic acid, caffeic acid, hydroxytyrosol acetate, verbascoside, deacetoxy-oleuropein aglycone, and/or cinnamyl glucoside.

Hydroxytyrosol is the main polyphenol found in olives and has the highest level of free radical protection activity ever reported for any natural antioxidant compound. If the additional compound is hydroxytyrosol, it can be present it various amounts. For example, in one embodiment, the hydroxytyrosol can be present in a ratio of hydroxytyrosol:oleocanthal in the range of 80:1 to 1:1. In another embodiment, the hydroxytyrosol can be present in a ratio of hydroxytyrosol:oleocanthal in the range of 60:1 to 1:1. Other exemplary ranges of ratios of hydroxytyrosol to oleocanthal are 40:1 to 1:1, 20:1 to 1:1, 10:1 to 1:1, and 5:1 to 1:1.

Similarly, tyrosol can also be present in the composition in different amounts. In one embodiment, the tyrosol can present in a ratio of tyrosol:oleocanthal in the range of 20:1 to 0.1:1. Other suitable ranges of ratios of tyrosol to oleocanthal include 10:1 to 0.1:1, 5:1 to 0.1:1, 1:1 to 0.1:1 and/or 0.5:1 to 0.1:1. Hydroxytyrosol acetate can also be present in various amounts in the composition. In some embodiments, hydroxytyrosol can be present in a ratio of hydroxytyrosol acetate:oleocanthal in the range of 30:1 to 1:1. In other embodiments, the hydroxytyrosol acetate can be present in a ratio of hydroxytyrosol acetate:oleocanthal in the range of 20:1 to 1:1, 10:1 to 1:1, 5:1 to 1:1, or 1:1 to 0.1:1.

Vanillic acid, 4-hydroxy-3-methoxybenzoic acid, is the oxidative form of vanillin. Various amounts of vanillic acid can be present in the composition. For example, in one embodiment, vanillic acid can be present in a ratio of vanillic acid:oleocanthal in the range of 10:1 to 0.1:1. Additional exemplary ranges of ratios of vanillic acid to oleocanthal include 5:1 to 0.1:1, 2:1 to 0.1:1, 1:1 to 0.1:1 and 0.5:1 to 0.1:1. Caffeic acid is known as a naturally occurring phenolic acid which acts as a carcinogenic inhibitor. In some embodiments, caffeic acid can be present in a ratio of caffeic acid:oleocanthal in the range of 1:1 to 0.01:1. Other suitable ranges of ratios of caffeic acid to oleocanthal include 0.5:1 to 0.01:1, 0.25:1 to 0.01:1, 0.1:1 to 0.01:1, and 0.05:1 to 0.01:1.

As with the other compounds, deacetoxy-oleuropein aglycone can also be present in various amounts in the composition. In one embodiment, deacetoxy-oleuropein aglycone can be present in a ratio of deacetoxy-oleuropein aglycone:oleocanthal in the range of 10:1 to 0.1:1. In another embodiment, deacetoxy-oleuropein aglycone can be present in a ratio of deacetoxy-oleuropein aglycone:oleocanthal in the range of 5:1 to 0.1:1. In other embodiments, deacetoxy-oleuropein aglycone can be present in a ratio of deacetoxy-oleuropein aglycone:oleocanthal in the ranges of 2:1:1 to 0.1:1, 1:1 to 0.1:1, or 0.5:1 to 0.1:1.

An exemplary composition contains oleocanthal; hydroxytyrosol that can be present in a ratio of about 21:1 hydroxytyrosol:oleocanthal; tyrosol that can be present in a ratio of about 4:1 tyrosol:oleocanthal; vanillic acid that can present in a ratio of about 2.5:1 vanillic acid:oleocanthal; caffeic acid can be present in a ratio of about 0.15:1 caffeic acid:oleocanthal; hydroxytyrosol acetate can be present in a ratio of about 10:1 hydroxytyrosol acetate:oleocanthal; and deacetoxy-oleuropein aglycone can be present in a ratio of about 1.5:1 deacetoxy-oleuropein aglycone:oleocanthal.

One method for producing a composition described herein is by the process illustrated below in Scheme 1.

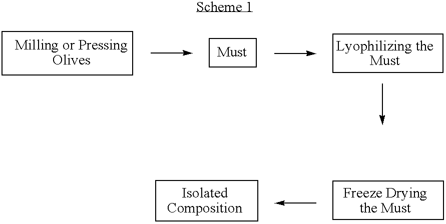

Any variety of olives can be used. One suitable variety of olives that can be used in this process is a leccino olive. In some of the embodiments, the composition produced by the process can include at least two, three, four, five, six, seven, eight, or nine of the compounds selected from the group consisting of oleocanthal, hydroxytyrosol, tyrosol, vanillic acid, caffeic acid, hydroxytyrosol acetate, verbascoside, deacetoxy-oleuropein aglycone, and cinnamyl glucoside. In one embodiment, the composition produced by the process can include oleocanthal. In another embodiment, the composition produced by the process can include oleocanthal and at least one other compound selected from the group consisting of a phytochemical, a stimulant, an antioxidant, and a secoiridoid. In another embodiment, the process can produce a composition that includes oleocanthal and at least one other compound selected from the group consisting of hydroxytyrosol, tyrosol, vanillic acid, caffeic acid, hydroxytyrosol acetate, verbascoside, deacetoxy-oleuropein aglycone, and cinnamyl glucoside. For example, the process can produce a composition that includes oleocanthal; hydroxytyrosol that can be in a ratio of about 21:1 hydroxytyrosol:oleocanthal; tyrosol that can be in a ratio of about 4:1 tyrosol:oleocanthal; vanillic acid that can be in a ratio of about 2.5:1 vanillic acid:oleocanthal; caffeic acid that can be a ratio of about 0.15:1 caffeic acid:oleocanthal; hydroxytyrosol acetate that can be in a ratio of about 10:1 hydroxytyrosol acetate:oleocanthal; and deacetoxy-oleuropein aglycone in a ratio of about 1.5:1 deacetoxy-oleuropein aglycone:oleocanthal.

One skilled in the art could vary the amounts of the compound(s) in the composition. For example, additional amounts of hydroxytyrosol could be added before freeze drying the must. Alternatively, one could add the additional amount of the desired compound(s) after freezing drying the must. If one desired to vary more than one of the amounts of the compounds in the composition, the additional amounts can be added separately or together. The amounts of the desired compounds(s) can be added by mixing, grinding, pressing, and/or any other suitable method known to one skilled in the art.

One of the embodiments described herein relates a nutraceutical or pharmaceutical composition that includes one of the compositions described herein and a pharmaceutically acceptable diluent and/or excipient. Pharmaceutical compositions for use in accordance with the present disclosure thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., as disclosed in Remington's Pharmaceutical Sciences. Mack Publishing Co., Easton, Pa., 18th edition, 1990, which is hereby incorporated by reference in its entirety.

Also, one of the embodiments described herein relates a nutraceutical composition that includes one of the compositions described herein and an acceptable diluent and/or excipient. An acceptable diluent and/or excipient includes those that are pharmaceutically acceptable, but also substances that do not rise to the level of being pharmaceutically acceptable, but which are safe for use in animals such as humans.

In selected embodiments, the compositions can include a microemulsion or nanoemulsion with ingredients including: non-reducing sugars, sugar polyols, or combinations thereof; modified starches; polysaccharides; glycerides selected from enzymatically modified oils, fats, and fatty acids of mono-, di-, and tri-glycerides; glycerides selected from lipolyzed modified oils, fats, and fatty acids of mono-, di-, and tri-glycerides; fruit concentrate sweetener as humectant that comprises a blend of hydrolyzed starch having a dextrose equivalent (D.E.) of up to approximately 25; fruit juice or fruit syrup concentrate of at least approximately 40% soluble solids and approximately 0% insoluble solids thereby forming a liquor having a dry weight composition of approximately 40 to approximately 65% complex carbohydrates; and approximately 35 to approximately 55% simple sugars from the fruit juice or fruit syrup concentrate; and approximately 0 to approximately 5% nutritional components occurring naturally in the fruit juice or fruit syrup concentrate; cocoa powder; Sucralose; and combinations thereof.

In other embodiments, the compositions may be made into products including: nutraceutical, or pharmaceutical products; products having vegetable oils including rice bran oil, flax, chia, olive hemp, castor, soybean, lesquerella, dehydrated castor oil, rich in Omega-3, or conjugated linoleic acid, animal oils including fish, egg, poultry, and beef oils rich in Omega-3, or conjugated linoleic acid, or combinations thereof; beverage products being transparent comprised of calcium and magnesium salts; cocoa products having improved creaminess, reduced bitterness, and reduced oxidation; protein rich products, comprised of high-methoxyl pectins or pectin alginates or combinations thereof having reduced protein settling and sedimentation; protein rich products having reduced protein settling and sedimentation; oil-in-water micro- and nano-emulsions having increased emulsion and oxidation stability; or water-in-oil micro- and nano-emulsions having increased emulsion and oxidation stability.

The range of products include, but are not limited to, confectionery, baked goods, spreads, dressings, salad dressings, nutraceutical supplements, functional foods products, ice cream, yogurts, seed milks, sports or health bars, dairy products, pharmaceutical tablets, a foodstuff that can be added to other foods for consumption, syrups, and medicines, functional confectionery products, and mineral-enriched drinks including carbonated and non-carbonated beverages and “sports drinks,” and powders that can be mixed with liquids to make enriched drinks. When making any of the compositions or products listed herein, the compositions and materials described herein can be mixed according to the skill in the particular arts and industries.

For oral administration, the compounds can be formulated readily by combining the active compounds with carriers, including pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active substances in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

Another embodiment described herein relates to a process for producing a composition that includes the steps of: (i) milling or pressing an olive (e.g., a leccino olive) to obtain a must; (ii) lyophilizing the must; and (iii) freeze drying the lyophilized must. In one embodiment, the composition can include oleocanthal. In another embodiment, the process can produce a composition that contains at least two, three, four, five, six, seven, eight, or nine of the compounds selected from the group consisting of oleocanthal, hydroxytyrosol, tyrosol, vanillic acid, caffeic acid, hydroxytyrosol acetate, verbascoside, deacetoxy-oleuropein aglycone, and cinnamyl glucoside. In still another embodiment, the process can produce a composition that includes oleocanthal and at least one other compound such as a phytochemical, an antioxidant, and/or a secoiridoid. For example, the composition produced by the process described herein can include oleocanthal and at least one other compound selected from the group consisting of hydroxytyrosol, tyrosol, vanillic acid, caffeic acid, hydroxytyrosol.

Some embodiments disclosed herein relate to a method of ameliorating or treating inflammation that includes administering to a subject in need a composition described herein. Other embodiments disclosed herein relate to a method of ameliorating or treating inflammation that includes administering to a subject in need a pharmaceutical composition described herein. Still another embodiment described herein relates to a method of ameliorating or treating inflammation comprising administering to a subject in need the composition obtained from the process for producing the composition described herein. These embodiments may further comprise identifying a subject that suffers from inflammation and/or has an inflammatory disease such as an autoimmune disease.

Some embodiments relate to the above described compositions and substance in combination with other medications, including, for example, other anti-inflammatory and pain agents. Examples include, ibuprofen, aspirin, acetaminophen, coriticosteroids, NSAIDs, and the like.

The compounds and compositions described herein may used in the amounts listed below or in any other amount that is effect to reduce inflammation or oxidation in an animal. The compounds and compositions can be orally or non-orally administered to a human patient alone or as a pharmaceutical, nutraceutical, food, beverage or other substance as described herein in the amount of about 0.001 mg/kg (per day or per serving/dosage) to about 10,000 mg/kg (per day or per serving/dosage) of the active ingredient, and more preferably about 0.1 mg/kg (per day or per serving/dosage) to about 100 mg/kg (per day or per serving/dosage) of the active ingredient. The compositions and compounds can be beneficially used for example, one time per day or, less preferably, over two to about ten times per day. Nonetheless, as will be understood by those of skill in the art, in certain situations it may be necessary to administer the compositions and compounds of the embodiment in amounts that excess, or even far exceed, the above-stated, preferred dosage range to effectively and aggressively treat particularly inflammatory or oxidation conditions.

The items that include the compositions and compounds (food items, beverages, nutraceuticals, powders, yogurts and dairy products, etc.) can include the compounds and compositions as a percentage of their total weight or volume, or in any amount that is effective for the intended nutritional or pharmaceutical purpose. For example, effective for reducing the inflammatory condition or effective to provide and antioxidant effect to the animal that uses the item. The percentages by weight or volume can be any include from 0.00001% to about 99%, preferably about 0.01% to about 75%, preferably in other embodiments 0.1% to about 50%, 1% to about 25%, and the like.

### EXAMPLES

### Example 1

Entire leccino olives were milled and pressed in a pilot plant that is made up of a hopper for charging olives, an Archimedian screw and a hammer mill for crushing the fruits. The resulting olive paste was distributed on steel superimposed stokers inside a drilled cylinder. A hydraulic piston press acts on the stokers which allowed for the dripping of the paste and the recovery of the must which is made up of oil and vegetation waters. The must was lyophilized with a pilot plane, and freeze dried.

### Example 2

A composition according to Example 1 is administered to a person suffering from an inflammatory condition in an amount that sufficient to reduce the inflammation.

### Example 3

A composition according to Example 1 is administered to a person suffering from a condition associated with oxidation or free radicals in an amount that sufficient to reduce the condition.

### Example 4

A composition is provided that includes oleocanthal and one or more of the following: hydroxytyrosol, tyrosol, vanillic acid, caffeic acid, hydroxytyrosol acetate, verbascoside, deacetoxy-oleuropein aglycone, and cinnamyl glucoside. The composition is administered to a person suffering from an inflammatory condition in an amount that sufficient to reduce the inflammation.

### Example 5

A composition is provided that includes oleocanthal and one or more of the following: hydroxytyrosol, tyrosol, vanillic acid, caffeic acid, hydroxytyrosol acetate, verbascoside, deacetoxy-oleuropein aglycone, and cinnamyl glucoside. The composition is administered to a person suffering from a condition associated with oxidation or free radicals in an amount that sufficient to reduce the condition.

Although the invention has been described with reference to the above example, it will be understood that modifications and variations are encompassed within the spirit and scope of the invention. Accordingly, the invention is limited only by the following claims.

What is claimed is:

1. A composition comprising oleocanthal and at least one other compound selected from the group consisting of a phytochemical, an antioxidant, and a secoiridoid or derivatives thereof.

2. A composition comprising oleocanthal and at least one other compound selected from the group consisting of hydroxytyrosol, tyrosol, vanillic acid, caffeic acid, hydroxytyrosol acetate, verbascoside, deacetoxy-oleuropein aglycone, and cinnamyl glucoside.

3. The composition of claim 2, wherein the hydroxytyrosol is present in a ratio of hydroxytyrosol:oleocanthal in the range of 60:1 to 1:1.

4. The composition of claim 3, wherein the hydroxytyrosol is present in a ratio of hydroxytyrosol:oleocanthal in the range of 40:1 to 1:1.

5. The composition of claim 4, wherein the hydroxytyrsol is present in a ratio of hydroxytyrosol:oleocanthal in the range of 20:1 to 1:1.

6. The composition of claim 2, wherein the tyrosol is present in a ratio of tyrosol:oleocanthal in the range of 10:1 to 0.1:1.

7. The composition of claim 6, wherein the tyrosol is present in a ratio of tyrosol:oleocanthal in the range of 5:1 to 0.1:1.

8. The composition of claim 2, wherein the vanillic acid is present in a ratio of vanillic acid:oleocanthal in the range of 10:1 to 0.1:1.

9. The composition of claim 8, wherein the vanillic acid is present in a ratio of vanillic acid:oleocanthal in the range of 5:1 to 0.1:1.

10. The composition of claim 9, wherein the vanillic acid is present in a ratio of vanillic acid:oleocanthal in the range of 2:1 to 0.1:1.

11. The composition of claim 2, wherein the caffeic acid is present in a ratio of caffeic acid:oleocanthal in the range of 1:1 to 0.01:1.

12. The composition of claim 11, wherein the caffeic acid is present in a ratio of caffeic acid:oleocanthal in the range of 0.5:1 to 0.01:1.

13. The composition of claim 12, wherein the caffeic acid is present in a ratio of caffeic acid:oleocanthal in the range of 0.25:1 to 0.01:1.

14. The composition of claim 2, wherein the hydroxytyrosol acetate is present in a ratio of hydroxytyrosol acetate:oleocanthal in the range of 20:1 to 1:1.

15. The composition of claim 14, wherein the hydroxytyrosol acetate is present in a ratio of hydroxytyrosol acetate:oleocanthal in the range of 10:1 to 1:1.

16. The composition of claim 2, wherein the deacetoxy-oleuropein aglycone is present in a ratio of deacetoxy-oleuropein aglycone:oleocanthal in the range of 10:1 to 0.1:1.

17. The composition of claim 16, wherein the deacetoxy-oleuropein aglycone is present in a ratio of deacetoxy-oleuropein aglycone:oleocanthal in the range of 5:1 to 0.1:1.

18. The composition of claim 17, wherein the deacetoxy-oleuropein aglycone is present in a ratio of deacetoxy-oleuropein aglycone:oleocanthal in the range of 2:1 to 0.1:1.

19. The composition of claim 2, wherein:
hydroxytyrosol is present in a ratio of about 21:1 hydroxytyrosol:oleocanthal;
tyrosol is present in a ratio of about 4:1 tyrosol:oleocanthal;
vanillic acid is present in a ratio of about 2.5:1 vanillic acid:oleocanthal;
caffeic acid is present in a ratio of about 0.15:1 caffeic acid:oleocanthal;
hydroxytyrosol acetate is present in a ratio of about 10:1 hydroxytyrosol acetate:oleocanthal; and
deacetoxy-oleuropein aglycone is present in a ratio of about 1.5:1 deacetoxy-oleuropein aglycone:oleocanthal.

20. A method of ameliorating or treating inflammation comprising administering to a subject in need an effective amount of a composition comprising oleocanthal, a pharmaceutically acceptable diluent or excipient, and at least one compound selected from the group consisting of a phytochemical, an antioxidant, and a secoiridoid or derivatives thereof.
